(19) United States
(12) Patent Application Publication
(10) Pub. No.: US 2013/0172679 A1
(43) Pub. Date: Jul. 4, 2013

(54) ENDOSCOPE INSERTION ASSISTING DEVICE

(71) Applicants: Tsuyoshi ASHIDA, Kanagawa (JP); Takayuki NAKAMURA, Kanagawa (JP); Shinichi YAMAKAWA, Kanagawa (JP); Yasunori OHTA, Kanagawa (JP); Maureen P. Sheridan, Eagan, MN (US)

(72) Inventors: Tsuyoshi ASHIDA, Kanagawa (JP); Takayuki NAKAMURA, Kanagawa (JP); Shinichi YAMAKAWA, Kanagawa (JP); Yasunori OHTA, Kanagawa (JP); Timothy P. SHERIDAN, Eagan, MN (US)

(73) Assignee: FUJIFILM CORPORATION, Tokyo (JP)

(21) Appl. No.: 13/538,458

(22) Filed: Jun. 29, 2012

Related U.S. Application Data
(60) Provisional application No. 61/503,994, filed on Jul. 1, 2011.

(51) Int. Cl. A61B 1/00; A61B 1/07; A61B 1/12; A61B 1/05; A61B 1/015; A61B 1/018; A61B 17/29; A61B 17/32; A61M 5/32; A61B 1/31; A61B 1/005

(52) U.S. Cl. CPC A61B 1/00156; A61B 1/00006; A61B 1/0016; A61B 1/31; A61B 1/00066; A61B 1/00114; A61B 1/07; A61B 1/12; A61B 1/051; A61B 1/0052; A61B 1/00096; A61B 1/00087; A61B 1/018; A61B 17/29; A61B 17/320016; A61M 5/32; A61B 1/015; A61B 1/00154
USPC 600/114

(57) ABSTRACT

If an instruction indicating advance is input, first and second motors are driven to rotate first and second torque wires. A rotary body is circulated by the rotation of the first and second torque wires, and a propelling unit advances. The first and second TGs detect the rotating speeds of the first and second torque wires. If disconnection of the first torque wire is detected by a disconnection detecting section, a motor control section stops driving of the first motor coupled to the disconnected first torque wire, and rotates the second motor coupled to the second torque wire that is not disconnected at a low speed to rotate the second torque wire at a low speed. Through the low-speed rotation of the second torque wire, the propelling unit advances at a low speed or retreats at a low speed.

### BACKGROUND OF THE INVENTION

1. Field of the Invention

The present invention relates to an endoscope insertion assisting device for inserting an insertion part of an endoscope within a test subject.

2. Description of the Related Art

Diagnosis using an endoscope is widely performed in a medical field and an industrial field. The endoscope includes a manipulating part, and an insertion part to be inserted into a test subject. An imaging device, such as CCD, is housed at a tip end of the insertion part, and an image captured by the imaging device is displayed on a monitor.

An endoscope insertion assisting device that facilitates insertion of an endoscope is known. For example, an endoscope insertion assisting device described in JP 2005-253892 A includes a tubular supporting body mounted on a tip end side of an insertion part, and a circulating body attached to the supporting body in a circulating manner. The rotary body is circulated in a state where the outside thereof is brought into contact with a test subject, for example, an inner wall of an alimentary canal of a human body. Thereby, the tip end side of the endoscope is self-propelled within the alimentary canal by the friction produced between the outside of the rotary body and the test subject. Accordingly, insertion of the endoscope can be easily performed even in an alimentary canal that is greatly curved, for example, like the large intestine.

In the endoscope insertion assisting device described in JP 2005-253892 A, the circulating body bridged over rollers made of magnets is moved in a circulating manner by rotating a wire by a motor and rotating a magnet attached to the front end of the wire. The wire may be disconnected due to metal fatigue caused by use. If the wire is disconnected, the circulating body cannot be circulated. Therefore, there is a problem in that it becomes difficult to move (namely, advance or retreat) the insertion part within a test subject.

### SUMMARY OF THE INVENTION

An object of the present invention is to provide an endoscope insertion assisting device capable of safely moving an insertion part after wire disconnection.

In order to achieve the above object, an endoscope insertion assisting device of the present invention includes a propelling unit, a plurality of wires, a plurality of motors, a disconnection detecting unit, and a control unit. The propelling unit has a mounting cylinder, a transmission gear cylinder, a plurality of rotary gears, a supporting cylinder, a circulating body, and a circulation driving section. The mounting cylinder is detachably mounted on a tip end portion of the insertion part. The transmission gear cylinder is rotatably fitted on an outside of the mounting cylinder. The plurality of rotary gears mesh with the transmission gear cylinder. The supporting cylinder is arranged outside the transmission gear cylinder. The circulating body is wound between an inner peripheral surface and an outer peripheral surface of the supporting cylinder and supported by the supporting cylinder so as to circulate along an axial direction of the mounting cylinder. The circulation driving section circulation drives the circulating body to circulate through the rotation of the transmission gear cylinder to propel the tip end portion within the test subject. The plurality of motors rotate the respective rotary gears via the respective wires. The disconnection detecting unit detects disconnection of the respective wires. When wire disconnection is detected by the disconnection detecting section, the control unit stops driving of a motor coupled to a disconnected wire and rotates a motor coupled to a wire that is not disconnected at a low speed.

Preferably, when the wire disconnection is detected, the control unit rotates the motors so that the insertion part is extracted out of the test subject.

Moreover, preferably, the endoscope insertion assisting device further includes a plurality of speed detectors. The plurality of speed detectors detect the rotating speeds of the respective wires. The disconnection detecting unit decides that a wire with a high rotating speed is disconnected when a difference between the rotating speeds of the respective wires detected by the respective speed detectors exceeds a preset value.

Preferably, the control unit includes a motor control section and a plurality of current supply sections. The motor control section instructs the rotating speeds of the motors. The plurality of current supply sections supply electric currents corresponding to the rotating speeds to the respective motors.

Preferably, when the wire disconnection is detected, the motor control section controls the current supply sections so that the motor coupled to the wire that is not disconnected rotates at a speed equal to or lower than a preset rotating speed.

Preferably, when the wire disconnection is detected, each of the current supply sections lowers the maximum value of an electric current to be supplied to the motor coupled to the wire that is not disconnected.

Moreover, preferably, the endoscope insertion assisting device further includes a plurality of current detecting sections. The plurality of current detecting sections detect electric currents when the respective motors are rotated. Each of the current supply sections calculates the rotating speed of each of the motors on the basis of the current value detected by each of the current detecting sections, and controls an electric current to be supplied to each of the motors so that the calculated rotating speed approximately coincides with the rotating speed instructed by the motor control section.

Preferably, the circulating body is a rotary body formed in the shape of a bag so as to cover the supporting cylinder over the entire circumference. Preferably, the circulating body is a plurality of endless belts that cover a part of the supporting cylinder in the circumferential direction.

Moreover, preferably, the endoscope insertion assisting device further includes a plurality of supporting rollers. The plurality of supporting rollers are rotatably attached to the supporting cylinder and brought into contact with the inner peripheral surface of the circulating body to support the circulating body in a circulating manner. The circulation driving section includes a worm gear and a plurality of driving gears. The worm gear is provided at the transmission gear cylinder. The plurality of driving gears mesh with the worm gear and pinch the circulating body between the driving gears and the plurality of supporting rollers to move the circulating body in a circulating manner.

According to the present invention, among the plurality of motors that drive the propelling unit, a motor coupled to a wire of which disconnection is detected is stopped, and a motor coupled to a wire that is not disconnected is rotated at a low speed. Thus, other wires can be prevented from being disconnected after one wire disconnection is detected. Thereby, the propelling unit can be inserted into and extracted out of a test subject even after wire disconnection.

### BRIEF DESCRIPTION OF THE DRAWINGS

The above object and advantages can be easily understood by those skilled in the art by reading the detailed description of the preferred embodiments of the present invention with reference to the attached drawings:

FIG. 1 is a side view showing an endoscope mounted with a propelling unit;

FIG. 2 is a block diagram showing an electrical configuration of an endoscope insertion assisting device;

FIG. 3 is a perspective view showing a state where the propelling unit is attached to a tip end side of an insertion part;

FIG. 4 is an exploded perspective view showing main parts of the propelling unit;

FIG. 5 is a cross-sectional view of the propelling unit;

FIG. 6 is a cross-sectional view taken along a line VI-VI of FIG. 5;

FIG. 7 is a cross-sectional view taken along a line VII-VII of FIG. 5;

FIG. 8 is a flowchart showing a procedure for inserting the endoscope into an alimentary canal;

FIG. 9 is a flowchart showing a procedure for insertion of the endoscope according to a second embodiment, in which a low motor rotating speed is instructed when wire disconnection is detected; and

FIG. 10 is a flowchart showing a procedure for insertion of the endoscope according to a third embodiment, in which the maximum value of an electric current to be supplied to a motor is lowered when wire disconnection is detected.

### DESCRIPTION OF THE PREFERRED EMBODIMENTS

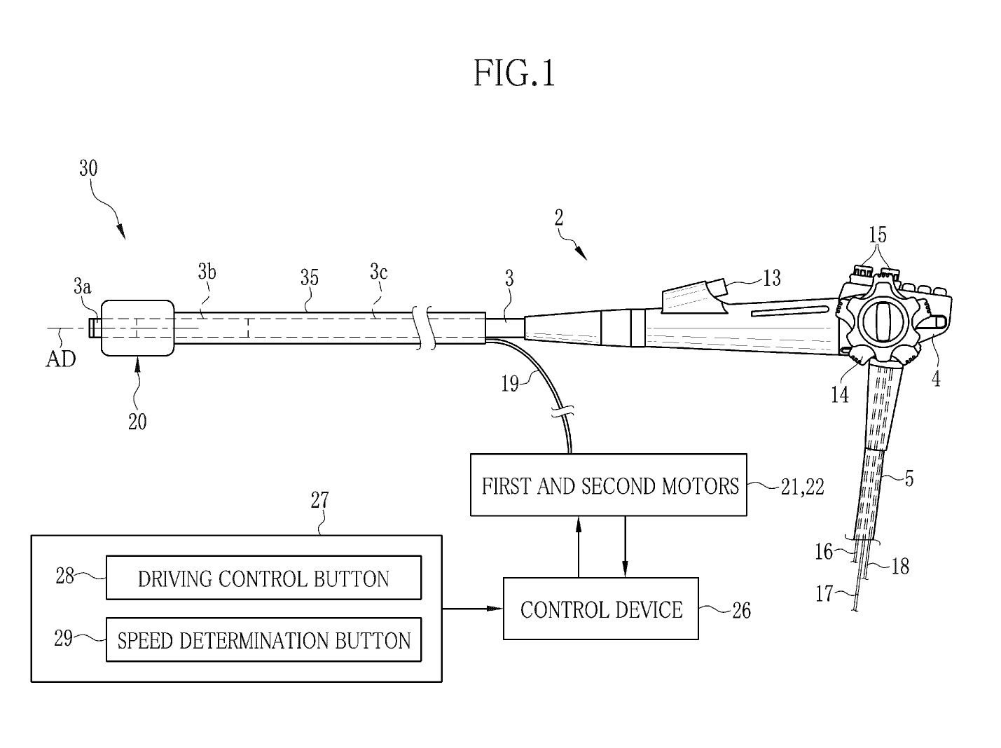

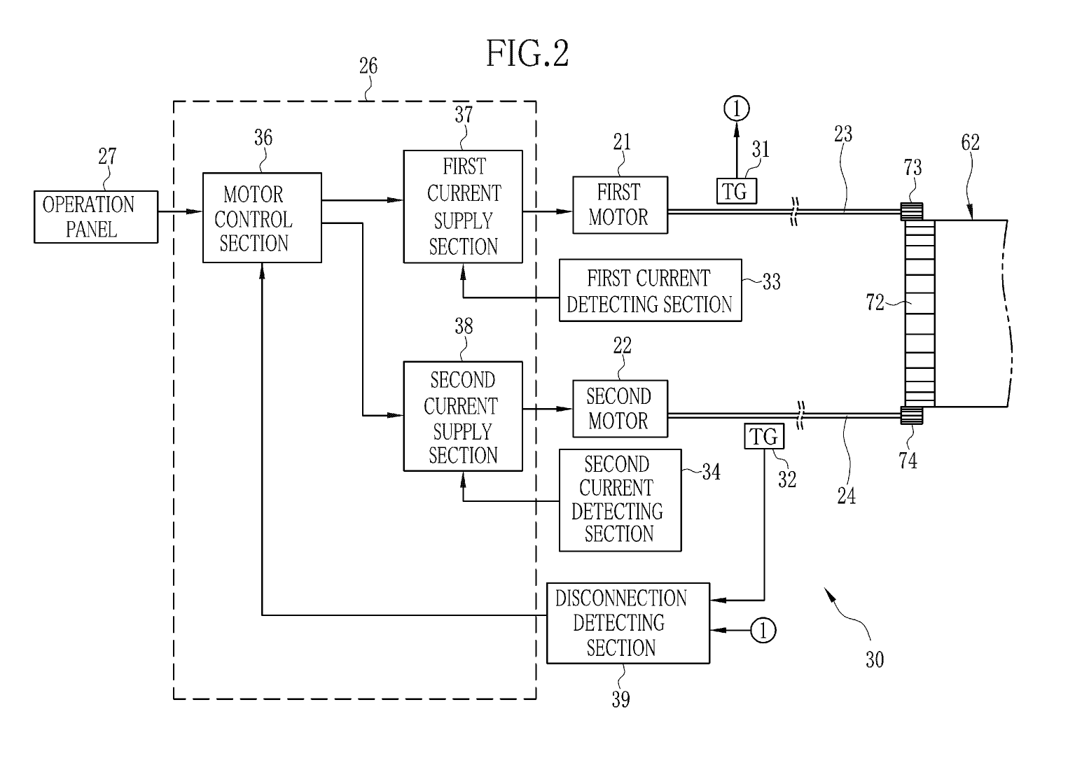

As shown in FIGS. 1 to 3, an endoscope 2 for medical use includes an insertion part 3 to be inserted into a test subject, for example, alimentary canals, such as the large intestine, a manipulating part 4 used to grip the endoscope 2 and to manipulate the insertion part 3, and a universal cord 5 for connecting the endoscope 2 to a processor device, a light source device, and an air/water supply device (none shown in the drawing).

The insertion part 3 includes a rigid tip end portion 3a having a solid-state image sensing device, for example, CCD sensor built therein, a curving portion 3b continuously provided at a rear end of the tip end portion 3a and capable of being curved in the up-and-down direction and in the right-and-left direction, and a flexible tube portion 3c continuously provided at the rear end of the curving portion 3b and having flexibility.

The tip end portion 3a of the insertion part 3 is provided with an imaging window 7, illumination windows 8a and 8b, and a forceps outlet 9 from which the front end of forceps protrudes. Additionally, the tip end portion 3a is provided with a jet nozzle 10 for jetting air or cleaning water toward the imaging window 7.

The illumination windows 8a and 8b are arranged on the both sides of the imaging window 7. The illumination light radiated from the light source device is irradiated toward the site to be observed in an alimentary canal through the illumination windows 8a and 8b. The illumination light reflected from the site to be observed enters a CCD sensor arranged behind the imaging window 7 through the imaging window 7.

The forceps outlet 9 communicates with a forceps entrance 13 provided in the manipulating part 4. Other than forceps, various treatment tools each having a syringe needle, a high-frequency knife, or the like disposed at its front ends are inserted into the forceps entrance 13.

The manipulating part 4 is provided with an angle knob 14 for curving the curving portion 3b in the up-and-down direction and in the right-and-left direction, and manipulation buttons 15 used for various manipulations, such as air supply, water supply, and suction.

The universal cord 5 is connected to the manipulating part 4. An air/water supply tube 16, an imaging signal cable 17, and a light guide 18 are housed in the universal cord 5. The air/water supply tube 16 has one end connected to the air/water supply device and the other end connected to the jet nozzle 10, and sends air or cleaning water supplied from the air/water supply device to the jet nozzle 10. The imaging signal cable 17 has one end connected to the processor device and the other end connected to the CCD sensor. The light guide 18 has one end connected to the illumination windows 8a and 8b and the other end connected to the light source device, and guides the illumination light radiated from the light source device to the illumination windows 8a and 8b.

An endoscope insertion assisting device 30 for advancing or retreating the insertion part 3 within an alimentary canal includes a propelling unit 20, a control device 26, an operation panel 27, and first and second motors 21 and 22. The propelling unit 20 is attached to the tip end side of the insertion part 3, and is driven by the respective motors 21 and 22. The first motor 21 is coupled to a first torque wire 23 for transmitting the rotary torque for propelling the propelling unit 20. Similarly, the second motor 22 is coupled to a second torque wire 24.

The respective torque wires 23 and 24 are inserted through the inside of a protective sheath 19. The respective torque wires 23 and 24 are rotated within the protective sheath 19 by the driving of the respective motors 21 and 22.

The respective motors 21 and 22 are controlled by the control device 26. The control device 26 is connected to the operation panel 27. The operation panel 27 includes driving control buttons 28 for inputting instructions for advance, retreat, and stop of the propelling unit 20, and a speed determination button 29 for determining the movement speed of the propelling unit 20. The operation panel 27 is operated by an operator (doctor).

An overtube 35 is externally fitted to the insertion part 3, and the protective sheath 19 is inserted between the overtube 35 and the insertion part 3.

The rotating speed of the first torque wire 23 is detected by a first tachometer generator (hereinafter referred to as TG) 31, and the rotating speed of the second torque wire 24 is detected by a second TG 32. Each of TGs 31 and 32 generates a direct current voltage proportional to the rotating speed, and sends the voltage to the control device 26. An electric current during the rotation of the first motor 21 is detected by a first current detecting section 33, and the electric current during the rotation of the second motor 22 is detected by a second current detecting section 34.

The control device 26 includes a motor control section 36 for controlling the rotational direction and speed of the respective motors 21 and 22, and a first current supply section 37 and a second current supply section 38 for supplying electric currents corresponding to the rotating speed instructed by the motor control section 36 to the respective motors 21 and 22. The motor control section 36 controls the respective motors 21 and 22 in accordance with instructions from the operation panel 27 and the disconnection information sent from the disconnection detecting section 39.

A current value detected by the first current detecting section 33 is input to the first current supply section 37, and an electric current to be supplied to the first motor 21 is feedback-controlled on the basis of the detected current value. Similarly, the second current supply section 38 feedback-controls an electric current to be supplied to the second motor 22 on the basis of a current value from the second current detecting section 34.

When the difference between the direct current voltage values sent from the TGs 31 and 32 exceeds a preset voltage value, the disconnection detecting section 39 decides that disconnection occurs. The disconnection occurs at a first rotary gear 73 coupled to the first torque wire 23 and a second rotary gear 74 coupled to the second torque wire 24, and therefore no load is imposed to the first and second torque wires 23 and 24. Consequently, the rotational speeds thereof become faster, and the direct current voltage values are increased. The direct current voltage values of the torque wires 23 and 24 are compared with each other, and one of the torque wires 23 and 24 with a higher direct current voltage value is decided to be disconnected. The motor control section 36 controls the driving of the respective motors 21 and 22 in accordance with a detection result in the disconnection detecting section 39. Furthermore, the actual rotating speeds of the respective motors 21 and 22 may be calculated on the basis of the respective direct current voltage values from the respective TGs 31 and 32, and the respective current supply sections 37 and 38 may be controlled so that the calculated rotating speeds approximately coincide with the rotating speeds instructed by the motor control section 36. In this case, the first and second current detecting sections 33 and 34 are omitted.

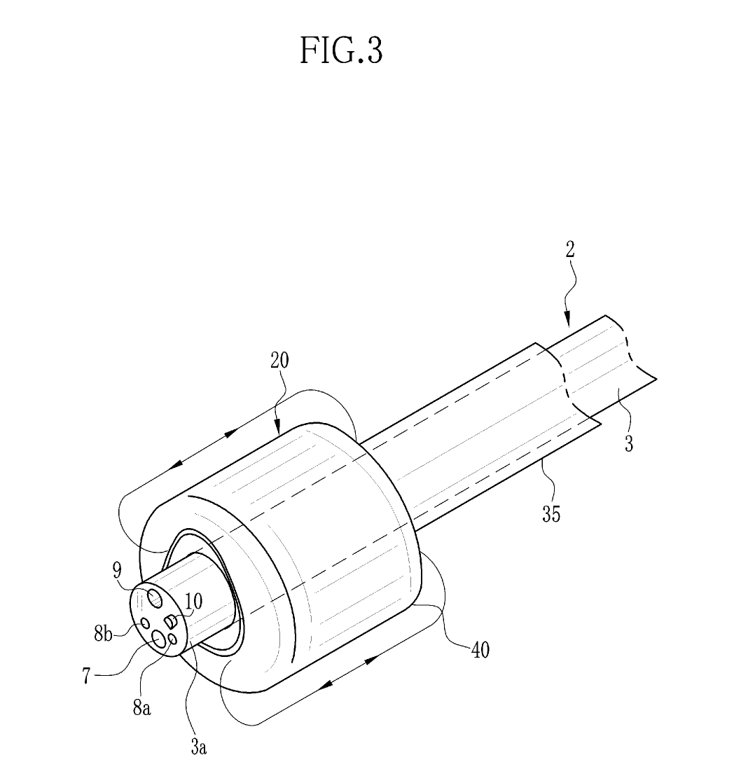

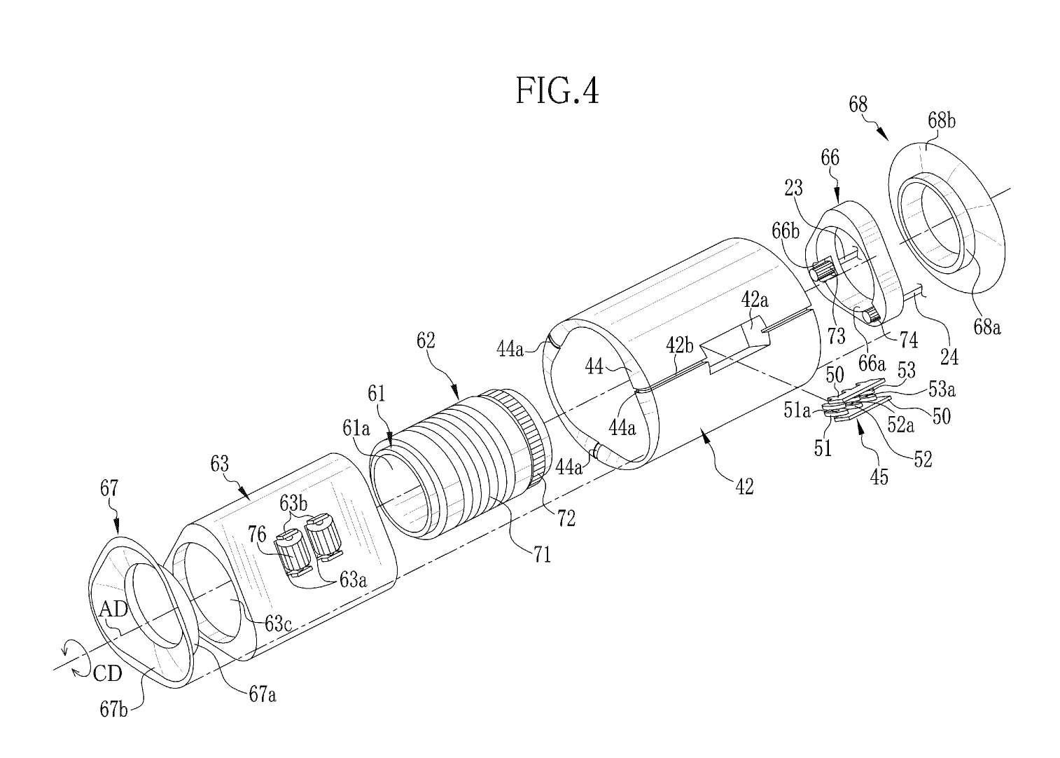

As shown in FIG. 3 and FIG. 4, the propelling unit 20 includes a rotary body in the shape of a doughnut (a toroid) 40 as a circulating body that comes into contact with the inner wall of an alimentary canal to generate a propulsive power in an insertion direction of the insertion part 3 of the endoscope 2 and in a retreat direction opposite to the insertion direction. The rotary body 40 is supported by a supporting cylinder (external cylinder) 42 so as to circulate in a direction along an axial direction AD, and covers over the entire circumference of the supporting cylinder 42. An arrow in FIG. 3 indicates the direction of the circulation of the rotary body 40. The rotary body 40 is formed from a material having flexibility (flexible material), and specifically, is preferably formed from biocompatible plastic such as polyvinyl chloride, polyamide resin, fluororesin, urethane, and polyurethane.

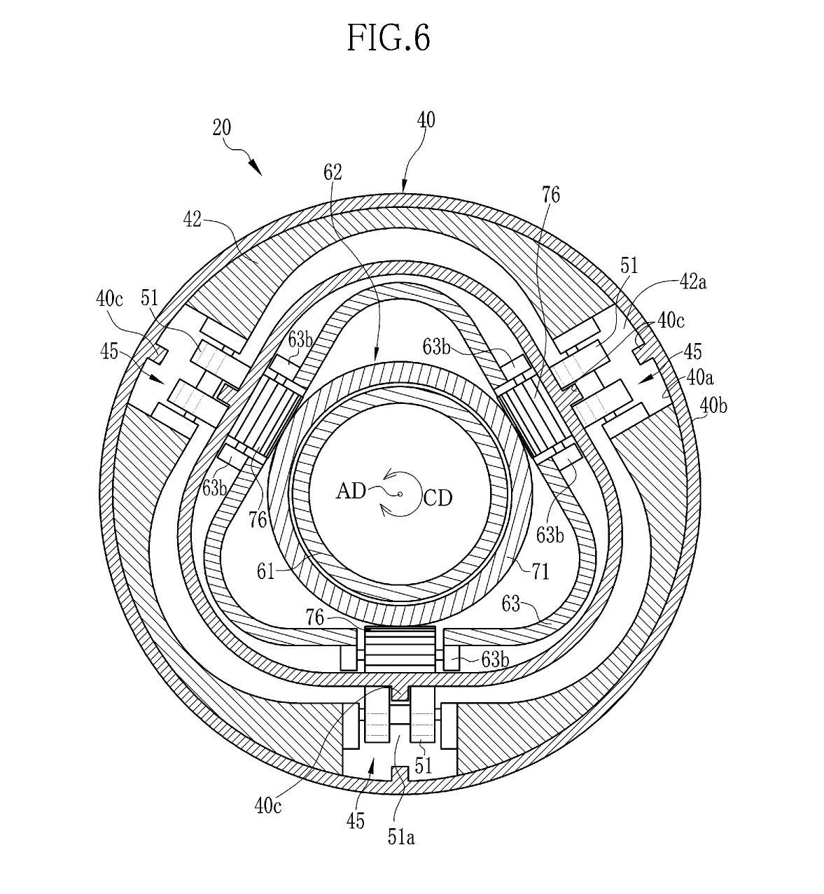

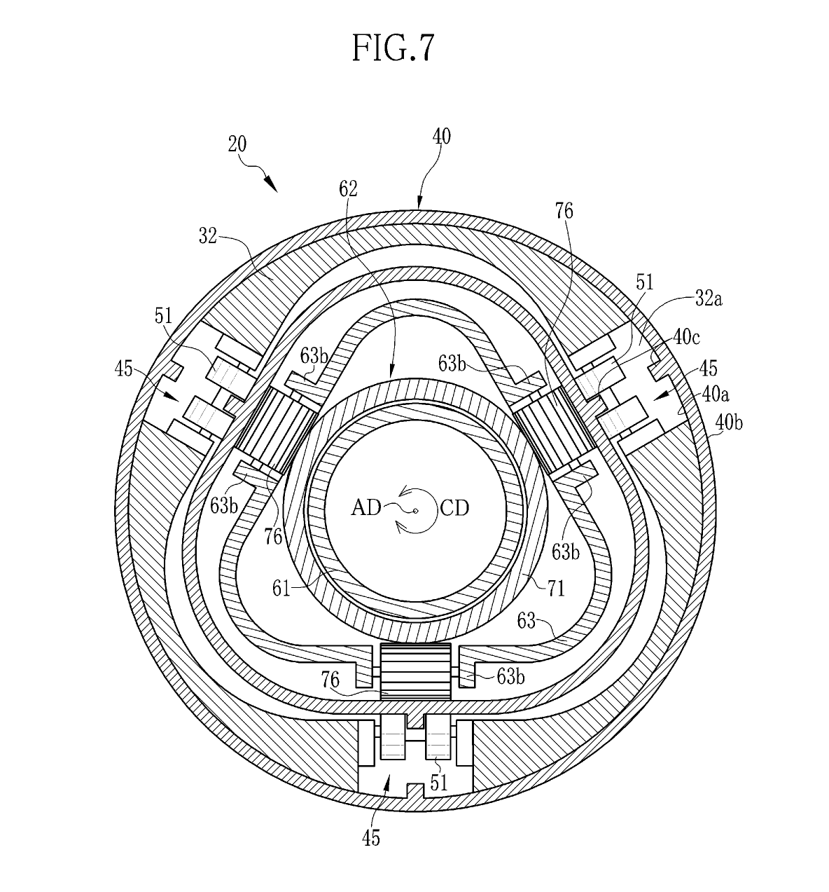

As shown in FIGS. 4 to 7, the supporting cylinder 42 has a tubular shape of which the cross-sectional shape in a direction orthogonal to the axial direction AD is circular on the outer peripheral surface and is substantially triangular (a shape such that each angle of an equilateral triangle is curved and rounded) on the inner peripheral surface, and the rotary body 40 is wound around the supporting cylinder 42.

The rotary body 40 developed in a sheet-like manner is inserted into the supporting cylinder 42, and then folded back along the shape of the supporting cylinder 42 to be wound therearound. After being wound around the supporting cylinder 42, both ends of the rotary body 40 are bonded together by heat welding or the like to make the rotary body 40 endless.

A ring-shaped contact body 44 that comes into contact with the folded-back portion of rotary body 40 is attached to each of front and rear ends of the supporting cylinder 42. The contact body 44 has a substantially semi-circular cross-section, and is made of a material that makes the rotary body 40 circulate smoothly (for example, nylon). Furthermore, the contact body 44 may be made of a material having high slidability such as PEEK and Teflon, without being limited to nylon.

Three straight-line portions are formed on the inner peripheral surface of the supporting cylinder 42. Openings 42a are formed in the three straight-line portions on one-to-one basis. A roller unit 45 for supporting the rotary body 40 such that the rotary body 40 can circulate is attached to each of the openings 42a. In the roller unit 45, first to third supporting rollers 51 to 53 are rotatably attached in order along the axial direction AD between two supporting plates 50. Furthermore, the respective rollers 51 to 53 may be directly attached to the supporting cylinder 42. The locations where the roller units 45 are attached are not limited to three locations, and the number of the roller units may be appropriately changed.

An inner surface 40a of the rotary body 40 comes into contact with the respective supporting rollers 51 to 53. The portions of the rotary body 40 that come into contact with the respective supporting rollers 51 to 53 are thicker than other portions thereof, and have rigidity higher than that of the other portions.

Roller grooves 51a to 53a are formed at central portions of the supporting rollers 51 to 53 on one-to-one basis. Three linear projections 40c extending along the axial direction AD are formed on the inner surface 40a of the rotary body 40. The linear projections 40c slidably engage with the roller grooves 51a to 53a, and prevent the rotary body 40 from moving in the circumferential direction CD. Similarly, the supporting cylinder 42 is formed with grooves 42b with which the linear projections 40c slidably engage, and the contact body 44 is formed with grooves 44a with which the linear projections 40c slidably engage. Furthermore, lubricant is applied between the linear projections 40c and the grooves 42b and 44a and between the linear projections 40c and the roller grooves 51a to 53a, in order to enhance the slidability therebetween.

A cylindrical mounting cylinder 61 mounted with the tip end portion 3a of the endoscope 2, a transmission gear cylinder 62 rotatably supported outside the mounting cylinder 61, and a housing cylinder 63 for housing the mounting cylinder 61 and the transmission gear cylinder 62 are arranged inside the supporting cylinder 42.

A lid 66 is attached to a rear end of the housing cylinder 63. A front stopper 67 for preventing entering of the inner wall of an alimentary canal is attached to the front end of the housing cylinder 63, and a rear stopper 68 is similarly attached to the lid 66.

The transmission gear cylinder 62 is fitted into the mounting cylinder 61, and rotates around the axial direction AD. The transmission gear cylinder 62 is formed with a worm gear 71 having a central axis along the axial direction AD, and a spur gear 72 at which a plurality of gears are arranged in the circumferential direction. The spur gear 72 is formed at a rear end portion of the transmission gear cylinder 62. The first rotary gear 73 coupled to the first torque wire 23 and the second rotary gear 74 coupled to the second torque wire 24 mesh with the spur gear 72. The respective rotary gears 73 and 74 are rotated by the respective torque wires 23 and 24, and the spur gear 72 is rotated by the rotations of rotary gears 73 and 74, so as to rotate the transmission gear cylinder 62. The respective torque wires 23 and 24 are inserted through insertion holes (not shown) formed in the rear stopper 68.

The housing cylinder 63 is formed in a substantially triangular tubular shape (a shape such that each angle of the equilateral triangle is curved and rounded), so as to house the supporting cylinder 42. Openings 63a are respectively formed in three straight-line portions of the housing cylinder 63 on one-to-one basis. Two driving gears 76 are arranged at each of the three openings 63a. The front surface of the housing cylinder 63 is formed with an opening 63c, and a front end of the mounting cylinder 61 is inserted into the opening 63c.

The driving gears 76 are rotatably attached to attachment ribs 63b formed on the housing cylinder 63. The driving gears 76 are disposed between the first supporting roller 51 and the second supporting roller 52 and between the second supporting roller 52 and the third supporting roller 53, respectively. Further, the respective driving gears 76 mesh with the worm gear 71 of the transmission gear cylinder 62, come into contact with an outer surface 40b of the rotary body 40, and pinch the rotary body 40 between the respective driving gears and the first to third supporting rollers 51 to 53. The respective driving gears 76 overlap the respective supporting rollers 51 to 53 in the radial direction of the supporting cylinder 42, and the rotary body 40 is curved in a wavelike fashion between the supporting rollers 51 to 53 and the driving gears 76. Note that, the driving gear 76 is a worm wheel, and the teeth height thereof is low so as not to damage the rotary body 40. The shape of the driving gear 76 is similar to that of a concave-convex roller.

The front stopper 67 consists of a ring-shaped insertion portion 67a inserted into the opening 63c of the housing cylinder 63, and a stopper portion 67b for preventing the inner wall of an alimentary canal from entering the inside of the propelling unit 20. The stopper portion 67b has a conical shape whose diameter increases as the distance from the insertion portion 67a increases. The cross-sectional shape thereof is the same shape as the inner peripheral surface of the supporting cylinder 42 (namely, substantially triangular shape). The cross-sectional of the stopper portion 67b is slightly smaller than that of the supporting cylinder 42.

The lid 66 is formed in the same shape as the housing cylinder 63 (namely, substantially triangular shape), and is formed with an opening 66a that fits into the rear end of the mounting cylinder 61. The lid 66 is formed with two recesses 66b that rotatably accommodate the rotary gears 73 and 74, respectively. The respective rotary gears 73 and 74 accommodated in the respective recesses 66b mesh with the spur gear 72 of the transmission gear cylinder 62. The respective torque wires 23 and 24 are coupled to the respective rotary gears 73 and 74 via holes (not shown) formed in the lid 66.

The rear stopper 68 has the same configuration as that of the front stopper 67, and includes a ring-shaped insertion portion 68a to be inserted into the opening 66a of the lid 66, and a stopper portion 68b.

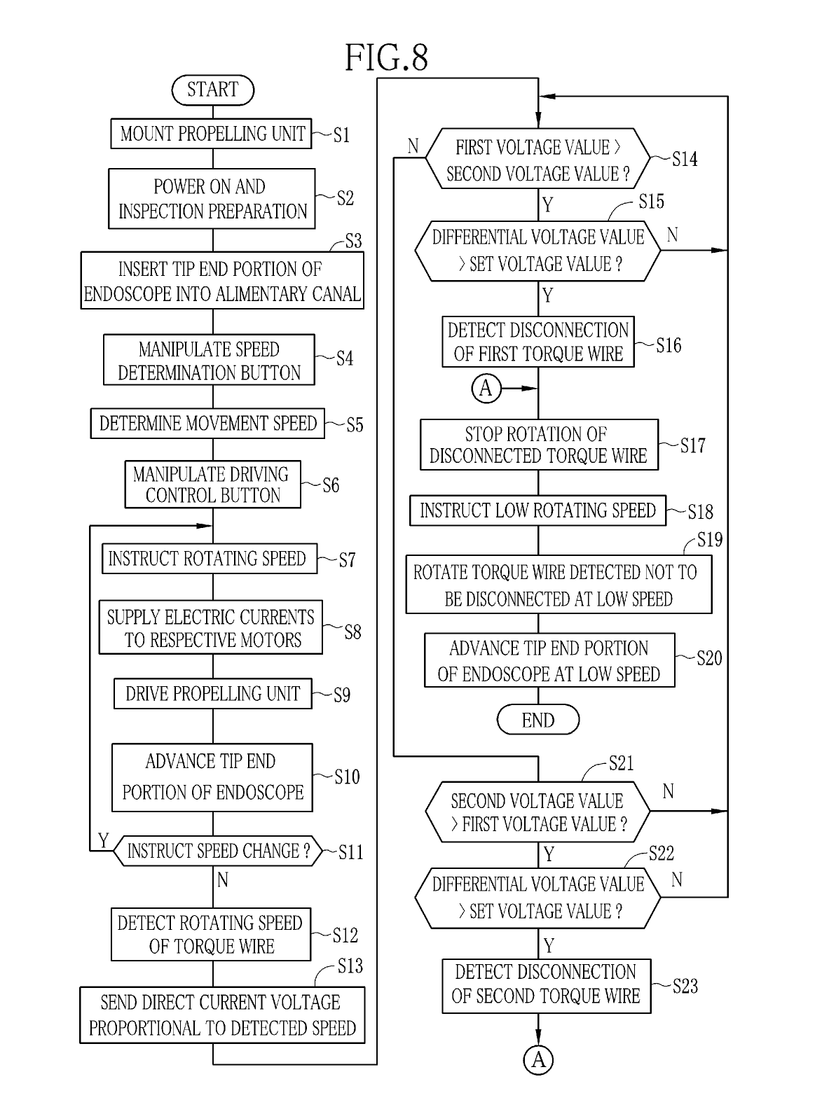

Next, the operation of the above embodiment will be described using a flowchart of FIG. 8. First, the tip end portion 3a of the endoscope 2 is inserted into an insertion hole 61a of the mounting cylinder 61, and the propelling unit 20 is mounted on the tip end side of the endoscope 20 (S1). Next, a power source for the processor, the light source device, the operation panel 27, and the like is turned on to perform inspection preparation (S2). After the inspection preparation is completed, the tip end portion 3a of the endoscope 2 is inserted together with the propelling unit 20 into an alimentary canal, for example, the large intestine of a patient (S3).

If the speed determination button 29 of the operation panel 27 is manipulated after the tip end portion 3a is advanced to a predetermined position within the large intestine, for example, a position just before the sigmoid colon (S4), the control device 26 determines the movement (insertion or retreat) speed of the propelling unit 20 (S5). Then, if the driving control button 28 is manipulated to input an instruction indicating advance (insertion) (S6), the motor control section 36 of the control device 26 instructs the rotating speeds of the respective motors 21 and 22 corresponding to the movement speed instructed by the speed determination button 29 to the first and second current supply sections 37 and 38 (S7). The movement speed of the propelling unit 20 may be determined before the tip end portion 3a is inserted into the large intestine.

The respective current supply sections 37 and 38 supply electric currents corresponding to the instructed rotating speeds to the respective motors 21 and 22 (S8). The respective motors 21 and 22 normally rotate in response to the supplied electric currents, and the first and second torque wires 23 and 24 are rotated in a normal direction. The spur gear 72 that meshes with the respective rotary gears 73 and 74 are rotated by the rotation of the respective rotary gears 73 and 74 accompanying the rotation of the first and second torque wires 23 and 24, and the transmission gear cylinder 62 is rotated.

The first current detecting section 33 detects the electric current of the first motor 21, and the detected current value is input to the first current supply section 37. The first current supply section 37 calculates the rotating speed of the first motor 21 on the basis of the input current value, and controls an electric current to be supplied to the first motor 21 so that the calculated rotating speed approximately coincides with the rotating speed instructed by the motor control section 36. Similarly, the second current supply section 38 controls an electric current to be supplied to the second motor 22 so that the calculated rotating speed of the second motor 22 approximately coincides with the rotating speed instructed by the motor control section 36. Here, “to approximately coincide” means to fall within a range of about ±20%.

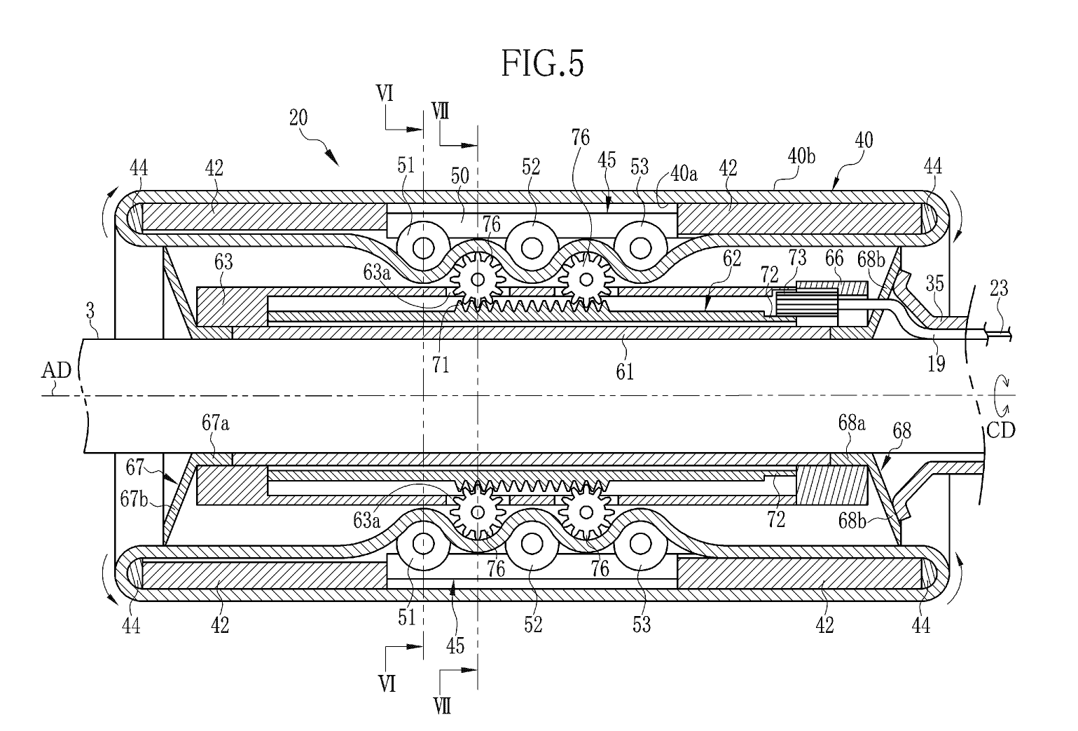

If the transmission gear cylinder 62 rotates, the driving gear 76 that meshes with the worm gear 71 rotates. The rotary body 40 pinched between the driving gears 76 and the first to third supporting rollers 51 to 53 is rotated in a direction shown by an arrow in FIG. 5 by the rotation of the driving gears 76 (S9). In accordance with the rotation of the rotary body 40, the outer surface 40b of the rotary body 40, which is located outside the supporting cylinder 42 and comes into contact with the inner wall of the large intestine, is moved in the retreat direction. The outer surface 40b of the rotary body 40 located inside the supporting cylinder 42 moves in the insertion direction, and thus the rotary body 40 circulates.

The rotary body 40 comes into contact with the inner wall of the large intestine, and moves in a circulating manner to generate an advancing force. The propelling unit 20 draws in the inner wall of the large intestine from the front side and sends it to the rear side by the advancing force, so as to advance the tip end portion 3a of the endoscope 2 along the inner wall of the alimentary canal (S10). On the other hand, in a case where the propelling unit 20 is propelled in the retreat direction, the rotary body 40 is moved in a circulating manner in the direction reverse to the above.

If the speed determination button 29 of the operation panel 27 is manipulated to instruct a speed change (Y in S11), the motor control section 36 instructs the rotating speeds of the respective motors 21 and 22 corresponding to the movement speed after the change to the current supply sections 37 and 38 (S7). Then, the respective current supply sections 37 and 38 supply electric currents corresponding to the instructed rotating speeds to the respective motors 21 and 22 (S8). Thereby, the rotating speeds of the respective motors 21 and 22 are changed, and the rotating speeds of the first and second torque wires 23 and 24 are changed, such that the movement speed of the propelling unit 20 is changed.

If the driving control button 28 of the operation panel 27 is manipulated to instruct retreat, the respective motors 21 and 22 are reversely rotated to reversely rotate the respective torque wires 23 and 24, thereby making the propelling unit 20 retreat. Moreover, if stop is instructed by manipulation of the driving control button 28, the rotation of each of the motors 21 and 22 is stopped. Since each of the respective torque wires 23 and 24 is stopped, the propelling unit 20 stops. By appropriately performing the above operations, the tip end portion 3a of the endoscope 2 can be propelled to a desired position within the large intestine.

The light from the light source device is radiated to the inside of the large intestine through the light guide 18 and the illumination windows 8a and 8b. The CCD built in the tip end portion 3a captures an image of the inside of the large intestine to output an imaging signal. The imaging signal is input to the processor device via the imaging signal cable 17 and the universal cord 5, and the image captured by the CCD is displayed on the monitor (not shown). The operator observes the inside of the large intestine through the monitor.

In a case where a lesion part is found during observation, a treatment tool suitable for the treatment of the lesion part is inserted into the forceps entrance 13. The treatment tool is protruded from the forceps outlet 9 so as to be used for the treatment of the lesion part. Ina case where the imaging window 7 is cleaned, the manipulation buttons 15 are manipulated to send the air or cleaning water supplied from the air/water supply device to the jet nozzle 10 via the air/water supply tube 16. The supplied air or cleaning water is jetted toward the imaging window 7 from the jet nozzle 10 to wipe away dirt such as body fluids adhering to the imaging window 7.

The first TG 31 detects the rotating speed of the first torque wire 23, and the second TG 32 detects the rotating speed of the second torque wire 24 (S12). Each of the TGs 31 and 32 generates a direct current voltage proportional to the detected rotating speed, and sends the value of the direct current voltage to the control device 26 (S13). The disconnection detecting section 39 of the control device 26 detects the presence or the absence of disconnection of the respective torque wires 23 and 24 on the basis of the direct current voltage values sent from the respective TGs 31 and 32.

In a case where the first torque wire 23 is disconnected, the rotating speed of the first torque wire 23 rises since the load imposed to the first torque wire 23 becomes small. Using the characteristics, in a case where a voltage value from the first TG1 31 (hereinafter referred to as a first voltage value) is higher than a voltage value from the second TG 32 (hereinafter referred to as a second voltage value) (Y in S14), and the difference between the first voltage value and the second voltage value (hereinafter referred to as a differential voltage value) exceeds a set voltage value (Y in S15), the disconnection detecting section 39 detects that the first torque wire 23 relating to the first voltage value is disconnected (S16).

If the disconnection of the first torque wire 23 is detected by the disconnection detecting section 39, the motor control section 36 performs switching from a normal mode in which there is no limitation to the driving of the propelling unit 20 to a low speed mode in which the propelling unit 20 is advanced or retreated at a speed lower than that of the normal mode.

If switching to the low speed mode is performed, the motor control section 36 stops the driving of the first motor 21 coupled to the disconnected first torque wire 23 via the first current supply section 37 (S17). Additionally, the motor control section 36 instructs the preset low rotating speed to the second current supply section 38 (S18). In response to this instruction, the second motor 22 is normally rotated at a low speed (a speed equal to or lower than the preset rotating speed) so as to rotate the second torque wire 24 at a low speed (S19). In accordance with the low-speed rotation of the second torque wire 24, the propelling unit 20 advances at a constant speed, and thereby the tip end portion 3a of the endoscope 2 advances at a low speed (S20) Additionally, in view of safety, in a case where the endoscope 2 is pulled out from the large intestine, the operation panel 27 is manipulated to instruct retreat. Consequently, since the second motor 22 reversely rotates at a low speed, the tip end portion 3a of the endoscope 2 is retreated at a low speed.

Accordingly, the propelling unit 20 is reliably driven even at the time of disconnection of the first torque wire 23. Moreover, since the transmission torque imposed to the second torque wire 24 is reduced in comparison with the time of high-speed rotation, the risk of disconnection of the second torque wire 24 is lowered. Furthermore, in the low speed mode, manipulation for determining the movement speed by the speed determination button 29 is disabled, so that the movement speed cannot be changed.

On the other hand, in a case where the second voltage value is higher than the first voltage value (N in S14 and Y in S21) and the differential voltage value exceeds a set voltage value (Y in S22), the disconnection detecting section 39 detects that the second torque wire 24 is disconnected (S23). If disconnection of the second torque wire 24 is detected by the disconnection detecting section 39, as mentioned above, the motor control section 36 performs switching from the normal mode to the low speed mode, and stops the driving of the second motor 22 coupled to the disconnected second torque wire 24 (S17). On the other hand, the first motor 21 is rotated at a constant low speed to rotate the first torque wire 23 (S19).

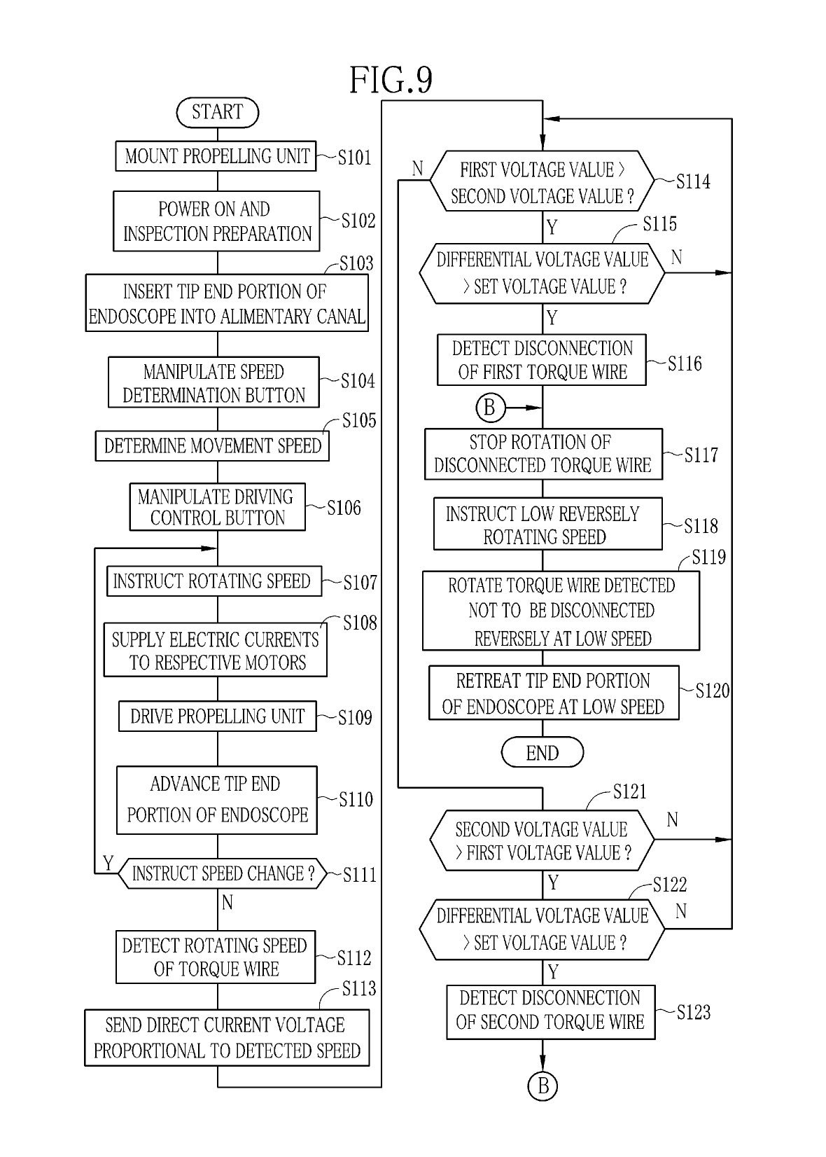

In a second embodiment shown in FIG. 9, a program of the motor control section 36 is changed to reversely rotate a motor at the time of detection of wire disconnection. Furthermore, since (S101) to (S115) are the same steps as (S1) to (S15) of the first embodiment, the description thereof is omitted.

If the disconnection of the first torque wire 23 is detected by the disconnection detecting section 39 (S116), the motor control section 36 performs switching from the normal mode to a low-speed retreat mode in which only retreat is performed at a lower speed than that of the normal mode. In the low-speed retreat mode, the motor control section 36 instructs the stop of the driving of the first motor 21 coupled to the disconnected first torque wire 23 to the first current supply section 37 (S117). Along with this, the motor control section 36 controls the second current supply section 38, such that the second motor 22 is reversely rotated at a speed equal to or lower than the preset rotating speed (S118). Through this control, the second motor 22 is reversely rotated at a low speed to reversely rotate the second torque wire 24 at a low speed (S119), and retreat the propelling unit 20 at a low speed. Thereby, even when the first torque wire 23 is disconnected, the tip end portion 3a of the endoscope 2 is retreated at a low speed and reliably extracted from the large intestine (S120).

In a case where the second voltage value is lower than the first voltage value (N in S114 and Y in S121) and the differential voltage value exceeds a set voltage value (Y in S122), the disconnection detecting section 39 detects that the second torque wire 24 is disconnected (S123). If disconnection of the second torque wire 24 is detected, as mentioned above, the motor control section 36 performs switching from the normal mode to the low-speed retreat mode, and stops the driving of the second motor 22 coupled to the disconnected second torque wire 24 (S117). Additionally, the first motor 21 connected to the first torque wire 23 that is not disconnected is reversely rotated at a low speed so as to reversely rotate the first torque wire 23 (S119) and retreat the tip end portion 3a of the endoscope 2 at a low speed (S120).

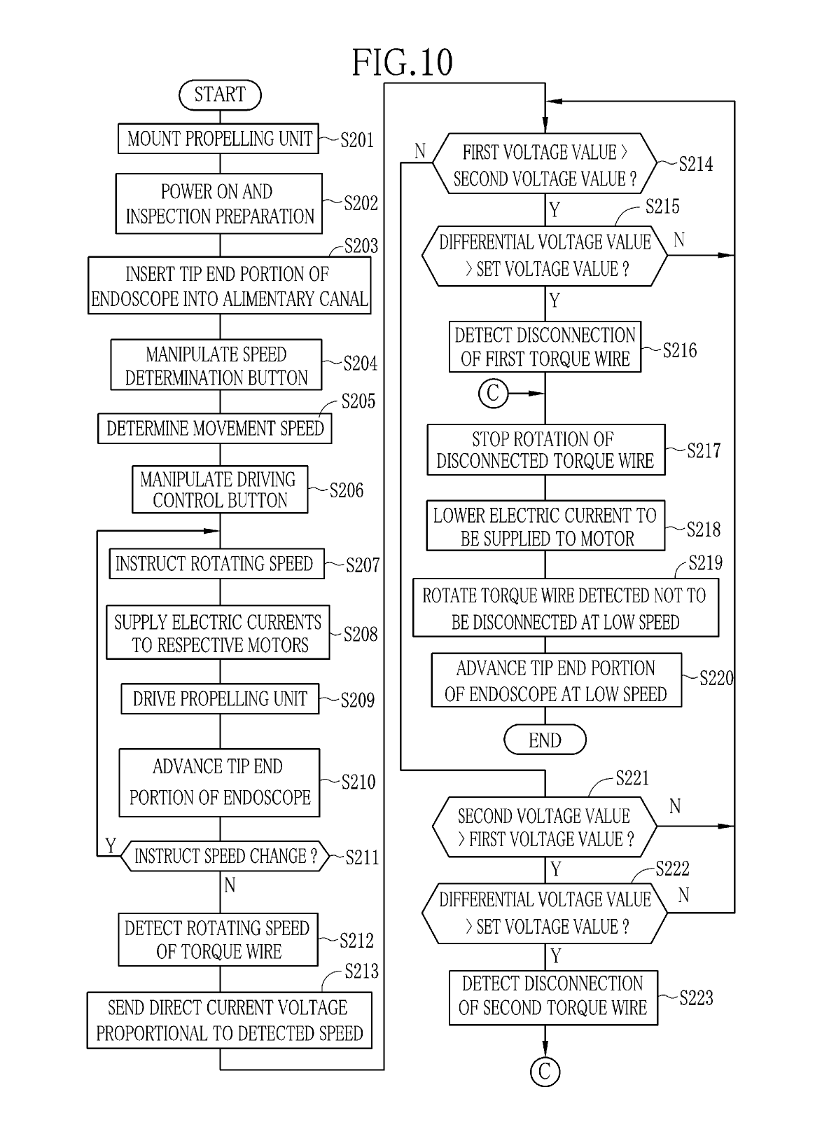

In a third embodiment shown in FIG. 10, the maximum values of electric currents to be supplied to the first and second motors 21 and 22 are lowered at the time of detection of wire disconnection. Furthermore, since (S201) to (S215) are the same steps as (S1) to (S15) of the first embodiment, the description thereof is omitted.

If the disconnection of the first torque wire 23 is detected by the disconnection detecting section 39 (S216), the motor control section 36 performs switching from the normal mode to a low current mode in which the maximum values of electric currents to be supplied to the respective motors 21 and 22 are lowered. In the low current mode, the motor control section 36 instructs the stop of the driving of the first motor 21 coupled to the disconnected first torque wire 23 to the first current supply section 37 (S217). Along with this, the motor control section 36 instructs the second current supply section 38 to lower the maximum value of an electric current to be supplied to the second motor 22 than that at the time of the normal mode (S218). The second current supply section 38 rotates the second motor 22 at a low speed to rotate the second torque wire 24 at a low speed (S219). In accordance with the low-speed rotation of the second torque wire 24, the propelling unit 20 advances at a low speed. Note that, the operation panel 27 is manipulated to instruct retreat, the propelling unit 20 is retreated (S219). Furthermore, in the low current mode, it is possible to select the movement direction of the propelling unit 20 and change the movement speed within the set maximum value of the electric current by manipulating the speed determination button 29. However, the manipulation for changing the movement speed by the speed determination button 29 is disabled, when the movement speed exceeds the set maximum value of the electric current.

On the other hand, in a case where the second voltage value is lower than the first voltage value (N in S214 and Y in S221) and the differential voltage value exceeds a set voltage value (Y in S222), the disconnection detecting section 39 detects that the second torque wire 24 relating to the second voltage value is disconnected (S223).

If the disconnection of the second torque wire 24 is detected by the disconnection detecting section 39, the motor control section 36 performs switching from the normal mode to the low current mode. In the low current mode, as mentioned above, the motor control section 36 rotates the first motor 21 at a low speed to rotate the first torque wire 23 at a low speed (S219). In accordance with the low-speed rotation of the first torque wire 23 that is not disconnected, the propelling unit 20 is advanced at a low speed to advance the tip end portion 3a of the endoscope 2 at a low speed (S220). Note that, it is also possible to instruct retreat.

In the above embodiments, the supporting cylinder is made to have a circular cross-section. However, any cylinder with a cross-section of polygons, such as a triangle and a quadrangle may be adopted.

Also, in the above embodiments, the endoscope is propelled by the rotary body that covers the supporting cylinder over the entire circumference as the circulating body. Additionally, a plurality of endless belts that cover a part of the supporting cylinder in the circumferential direction may be used. In this case, the endless belt may be directly driven by the worm gear. In a case where the driving gears are not provided, the rotational direction of the worm gear for advance or retreat becomes reverse in comparison with the case where the driving gears are provided. Therefore, it is necessary to change the instruction for advance or retreat by the manipulation unit in accordance with the presence or the absence of the driving gears.

In the above embodiments, the present invention is applied to an endoscope for medical diagnosis. However, the present invention is not limited to the application for medical diagnosis and can also be applied to other endoscopes, probes, or the like for industrial use or the like.

In the present invention, various alterations and modifications can be made without departing from the spirit of the present invention and such alterations and modifications should also be interpreted as being included in the scope of protection of the present invention.

What is claimed is:

1. An endoscope insertion assisting device for assisting insertion of an insertion part of an endoscope into a test subject, comprising:
a propelling unit having a mounting cylinder detachably mounted on a tip end portion of the insertion part, a transmission gear cylinder rotatably fitted on an outside of the mounting cylinder, a plurality of rotary gears that mesh with the transmission gear cylinder to rotate the transmission gear cylinder, a supporting cylinder arranged outside the transmission gear cylinder, a circulating body wound between an inner peripheral surface and an outer peripheral surface of the supporting cylinder and supported by the supporting cylinder so as to circulate along an axial direction of the mounting cylinder, and a circulation driving section that drives the circulating body to circulate through the rotation of the transmission gear cylinder to propel the tip end portion within the test subject;
a plurality of rotatable wires coupled to the respective rotary gears;
a plurality of motors coupled to the respective wires;
a disconnection detecting unit for detecting disconnection of the respective wires; and
a control unit for stopping driving of a motor coupled to a disconnected wire and rotating a motor coupled to a wire that is not disconnected at a low speed, when wire disconnection is detected by the disconnection detecting section.

2. The endoscope insertion assisting device according to claim 1, wherein when the wire disconnection is detected, the control unit rotates the motor coupled to the wire that is not disconnected so that the insertion part is extracted out of the test subject.

3. The endoscope insertion assisting device according to claim 2, further comprising a plurality of speed detectors for detecting the rotating speeds of the respective wires,
wherein when the difference between the rotating speeds of the respective wires detected by the respective speed detectors exceeds a preset value, the disconnection detecting unit decides that a wire with a high rotating speed is disconnected.

4. The endoscope insertion assisting device according to claim 3, wherein the control unit includes:
a motor control section for instructing the rotating speeds of the motors; and
a plurality of current supply sections for supplying electric currents corresponding to the rotating speeds to the respective motors.

5. The endoscope insertion assisting device according to claim 4, wherein when the wire disconnection is detected, the motor control section controls the current supply sections so that the motor coupled to the wire that is not disconnected rotates at a speed equal to or lower than a preset rotating speed.

6. The endoscope insertion assisting device according to claim 5, wherein when the wire disconnection is detected, the current supply section lowers the maximum value of an electric current to be supplied to the motor coupled to the wire that is not disconnected.

7. The endoscope insertion assisting device according to claim 6, further comprising a plurality of current detecting sections for detecting electric currents when the respective motors are rotated,
wherein each of the current supply sections calculates the rotating speed of each of the motors on the basis of the current value detected by each of the current detecting sections, and controls an electric current to be supplied to each of the motors so that the calculated rotating speed approximately coincides with the rotating speed instructed by the motor control section.

8. The endoscope insertion assisting device according to claim 7, wherein the circulating body is a rotary body formed in the shape of a bag so as to cover the supporting cylinder over the entire circumference.

9. The endoscope insertion assisting device according to claim 7, wherein the circulating body is a plurality of endless belts that cover a part of the supporting cylinder in the circumferential direction.

10. The endoscope insertion assisting device according to claim 8, further comprising a plurality of supporting rollers rotatably attached to the supporting cylinder and brought into contact with the inner peripheral surface of the circulating body so as to support the circulating body in a circulating manner,
wherein the circulation driving section includes:
a worm gear provided at the transmission gear cylinder; and
a plurality of driving gears that mesh with the worm gear and pinch the circulating body between the driving gears and the plurality of supporting rollers to drive the circulating body to move in a circulating manner.
